SEQUENCE LISTING (31 sequences)

SEQ ID NO: 1 - protein, 7 aa, Artificial Sequence
  Synthetic Sequence
GFSLTRY

SEQ ID NO: 2 - protein, 6 aa, Artificial Sequence
  Synthetic Sequence
IWGDGT

SEQ ID NO: 3 - protein, 13 aa, Artificial Sequence
  Synthetic Sequence
TRTGGYYDYVMDY

SEQ ID NO: 4 - protein, 15 aa, Artificial Sequence
  Synthetic Sequence
RASESVEYYVTSLMQ

SEQ ID NO: 5 - protein, 7 aa, Artificial Sequence
  Synthetic Sequence
AASNVES

SEQ ID NO: 6 - protein, 9 aa, Artificial Sequence
  Synthetic Sequence
QQSRKVPWT

SEQ ID NO: 7 - protein, 15 aa, Artificial Sequence
  Synthetic Sequence
RASKSVDSYGNSLMQ

SEQ ID NO: 8 - protein, 7 aa, Artificial Sequence
  Synthetic Sequence
GFSLSRY

SEQ ID NO: 9 - protein, 25 aa, Artificial Sequence
  Synthetic Sequence
QVTLRESGPALVKPTQTLTLTCTVS

SEQ ID NO: 10 - protein, 18 aa, Artificial Sequence
  Synthetic Sequence
GVSWIRQPPGKALEWLAV

SEQ ID NO: 11 - protein, 41 aa, Artificial Sequence
  Synthetic Sequence
TNYHSALISRLTISKDTSKNQVVLTMTNMDPVDTATYYCAG

SEQ ID NO: 12 - protein, 11 aa, Artificial Sequence
  Synthetic Sequence
WGQGSLVTVSS

SEQ ID NO: 13 - protein, 23 aa, Artificial Sequence
  Synthetic Sequence
DIVMTQSPDSLSVSLGERATINC

SEQ ID NO: 14 - protein, 15 aa, Artificial Sequence
  Synthetic Sequence
WYQQKPGQPPKLLIY

SEQ ID NO: 15 - protein, 32 aa, Artificial Sequence
  Synthetic Sequence
GVPDRFSGSGSGTDFTLTISSLQAEDVAVYYC

SEQ ID NO: 16 - protein, 10 aa, Artificial Sequence
  Synthetic Sequence
FGGGTKVEIK

SEQ ID NO: 17 - protein, 25 aa, Artificial Sequence
  Synthetic Sequence
QVTLKESGPTLVKPTQTLTLTCTFS

SEQ ID NO: 18 - protein, 41 aa, Artificial Sequence
  Synthetic Sequence
TNYHSALISRLTITKDTSKNQVVLTMTNMDPVDTATYYCAR

SEQ ID NO: 19 - protein, 41 aa, Artificial Sequence
  Synthetic Sequence
TNYNSALKSRLTISKDTSKNQVVLTMTNMDPVDTATYYCAG

SEQ ID NO: 20 - protein, 11 aa, Artificial Sequence
  Synthetic Sequence
WGQGTLVTVSS

SEQ ID NO: 21 - protein, 23 aa, Artificial Sequence
  Synthetic Sequence
DIVMTQSPDSLAVSLGERATINC

SEQ ID NO: 22 - protein, 10 aa, Artificial Sequence
  Synthetic Sequence
FGQGTKVEIK

SEQ ID NO: 23 - protein, 25 aa, Artificial Sequence
  Synthetic Sequence
  misc_feature (5)..(5): Xaa can be any naturally occurring amino acid
  misc_feature (10)..(10): Xaa can be any naturally occurring amino acid
  misc_feature (24)..(24): Xaa can be any naturally occurring amino acid
QVTLXESGPXLVKPTQTLTLTCTXS

SEQ ID NO: 24 - protein, 11 aa, Artificial Sequence
  Synthetic Sequence
  misc_feature (5)..(5): Xaa can be any naturally occurring amino acid
WGQGXLVTVSS

SEQ ID NO: 25 - protein, 11 aa, Artificial Sequence
  Synthetic Sequence
  misc_feature (5)..(5): Xaa can be any naturally occurring amino acid
WGQGXLVTVSS

SEQ ID NO: 26 - protein, 23 aa, Artificial Sequence
  Synthetic Sequence
  misc_feature (12)..(12): Xaa can be any naturally occurring amino acid
DIVMTQSPDSLXVSLGERATINC

SEQ ID NO: 27 - protein, 10 aa, Artificial Sequence
  Synthetic Sequence
  misc_feature (3)..(3): Xaa can be any naturally occurring amino acid
FGXGTKVEIK

SEQ ID NO: 28 - protein, 7 aa, Artificial Sequence
  Synthetic Sequence
  misc_feature (5)..(5): Xaa can be any naturally occurring amino acid
GFSLXRY

SEQ ID NO: 29 - protein, 42 aa, Artificial Sequence
  Synthetic Sequence
  misc_feature (4)..(4): Xaa can be any naturally occurring amino acid
  misc_feature (8)..(8): Xaa can be any naturally occurring amino acid
  misc_feature (14)..(14): Xaa can be any naturally occurring amino acid
  misc_feature (42)..(42): Xaa can be any naturally occurring amino acid
TNYXSALXSRLTIXLKDTSKNQVVLTMTNMDPVDTATYYCAX

SEQ ID NO: 30 - protein, 7 aa, Artificial Sequence
  Synthetic Sequence
  misc_feature (5)..(5): Xaa can be any naturally occurring amino acid
GFSLXRY

SEQ ID NO: 31 - protein, 41 aa, Artificial Sequence
  Synthetic Sequence
  misc_feature (14)..(14): Xaa can be any naturally occurring amino acid
  misc_feature (41)..(41): Xaa can be any naturally occurring amino acid
TNYHSALISRLTIXKDTSKNQVVLTMTNMDPVDTATYYCAX

(19) United States
(12) Patent Application Publication
(10) Pub. No.: US 2023/0138520 A1
(43) Pub. Date: May 4, 2023

(54) HUMANIZED ANTIBODIES SPECIFIC FOR MYELOMA AND OVARIAN CANCER CELLS

(71) Applicant: CAERUS THERAPEUTICS, INC., Manassas, VA (US)

(72) Inventor: Cohava GELBER, Nokesville, VA (US)

(73) Assignee: CAERUS THERAPEUTICS, INC., Manassas, VA (US)

(21) Appl. No.: 17/793,603

(22) PCT Filed: Jan. 14, 2021
(86) PCT No.: PCT/US21/13423

§ 371 Date: Jul. 18, 2022

Related U.S. Application Data
(60) Provisional application No. 62/961,768, filed on Jan. 16, 2020.

(51) Int. Cl. C07K 16/30; A61P 35/00; G01N 33/574; C12N 15/63

(52) U.S. Cl. CPC C07K 16/30; A61P 35/00; G01N 33/574; C12N 15/63; C07K 2317/565; C07K 2317/24

(57) ABSTRACT

The disclosure provides for humanized antibodies, and fragments thereof, that are capable of binding to and killing human myeloma and ovarian cancer cells. The antibodies and their fragments are useful for therapeutic, diagnostic, and research purposes.

### BACKGROUND OF THE INVENTION

Myeloma is a cancer of plasma cells in the bone marrow. Signs and symptoms of myeloma include anemia, fatigue, bone pain, kidney damage or failure, weight loss, fever, infections, blood clots, and hypercalcemia. An estimated 30,000 adults were diagnosed with myeloma and an estimated 12,000 to 13,000 adults died of myeloma in 2015.

Ovarian cancer ranks fifth in cancer deaths among women and it accounts for more cancer deaths in women than any other of the female reproductive system. In the United States alone it is estimated that approximately 23,000 women were diagnosed with and approximately 14,000 died from ovarian cancer in 2015. Women with ovarian cancer may experience pain in the abdomen or pelvis, bloating indigestion, nausea, weight loss, and fatigue.

An antibody capable of specifically binding to antigens on myeloma and ovarian cells, and of inhibiting their ability to divide and spread, would be highly beneficial to myeloma and ovarian cancer patients. An example of an antibody possessing these characteristics is murine antibody VAC69. Murine antibody VAC69 and its characterization are disclosed in U.S. Pat. No. 6,376,654, incorporated herein by reference for all purposes.

The epitope on human myeloma cells to which VAC69 binds is expressed on a single, glycosylated polypeptide with a molecular weight of about 78 kDa to about 120 kD, as determined by SDS PAGE under reducing conditions. The epitope on human ovarian cancer cells to which VAC69 binds is expressed as part of an antigen that is a single, glycosylated polypeptide with a molecular weight of about 76 kDa to about 213 kDa as determined by SDS PAGE under reducing conditions.

VAC69 was specific in its recognition of antigens on human myeloma and ovarian cancer cells; it did not bind to human peripheral blood mononuclear cells, human B cells, or human chronic myelogenic leukemia cells. VAC69 also did not react with any of an array of other human cancer cells such as lung, prostate, breast, cervical, neuroblastoma, lymphoma and leukemia. Further, VAC69 did not detect its antigen in human normal tissues, such as those derived from breast, ovary, prostate, colon, or lung.

The murine, VAC69 antibody, did not only specifically recognize human myeloma and ovarian cancer cells, it also induced the killing of the human myeloma and ovarian cancer cells in in vitro studies and in a SCID mouse model. Humanized VAC69 antibodies have been prepared that share the ability of VAC69 to recognize and bind to an epitope of an antigen expressed on human myeloma and ovarian cancer cells. The humanized antibodies also exhibited the ability to kill cancer cells via antibody dependent cellular toxicity. The humanized antibodies do have a further advantage over the VAC69 murine antibodies. Because they are humanized, they are less antigenic in humans and are therefore a more suitable, new, and useful therapeutic agent for treatment of human cancers.

### BRIEF SUMMARY OF THE INVENTION

The disclosure provides for antibodies and for fragments of antibodies that specifically recognize an epitope present on myeloma and ovarian cancer cells.

The disclosure further provides for methods that employ the antibodies and the fragments of the antibodies, e.g., methods of detecting myeloma or ovarian cancer cells.

### BRIEF DESCRIPTION OF FIGURES

FIG. 1. Alignment of humanized antibody variable heavy chain amino acid sequences. The sequence of individual antibody heavy chain CDR and framework sequences are separately noted, as are consensus sequences of humanized antibodies having similar CDR and framework sequences.

FIG. 2. Alignment of humanized antibody variable light chain amino acid sequences. The sequence of individual antibody light chain CDR and framework sequences are separately noted, as are consensus sequences of humanized antibodies having similar CDR and framework sequences.

FIG. 3. Alignment of the humanized antibody constructs (CD1-CD9), parental murine antibody (mVAC69) and a human/murine chimeric antibody (CD0) based on VAC69. Alignment of variable heavy (top) and variable light (bottom) chains are provided in CDR domains from two different references (Oak Biosciences and Abysis Chothia) are provided in overlaying color schema.

FIG. 4A and FIG. 4B. Determination of binding affinity of humanized antibody constructs via competition binding. FIG. 4A: U266 cells were cultured with each antibody (CD0-CD9 or mVAC69 (unlabeled)) at concentrations ranging from 1.36E-8 to 2.12E-10 moles. Fluorescently-labeled mVAC69 was then added to the tubes and binding affinity of this fluorescent probe was measured by flow cytometry. Percent inhibition was determined as the amount of antibody blocked from binding to the U266 cell (unlabeled minus sample fluorescence). FIG. 4B: Ki values. To estimate Ki (inhibition constant), the slope of the dilution effect was calculated and IC50/Ki determined as described (at the website tools-dot-thermofisher-dot-com-backslash-downloads-backslash-FP7-dot-pdf).

FIG. 5A and FIG. 5B. Humanized antibodies have ADCC activity as measured in a Promega ADCC Reporter Bioassay. FIG. 5A: U266 cells were cultured with Promega reporter cells and various antibodies at titrations ranging from 0.1-12.S μg/mL for 18 hours. FIG. 5B: EC₅₀ values. For the EC₅₀ values, log-transformed, normalized ADCC values provided calculations for the half-maximal response from the most active sample (CD0 (12.5 ng/ml, chimeric VAC69) using the linear trendline equation.

FIG. 6. FIG. 6 provides the amino acid sequences of SEQ ID NOs:1-31

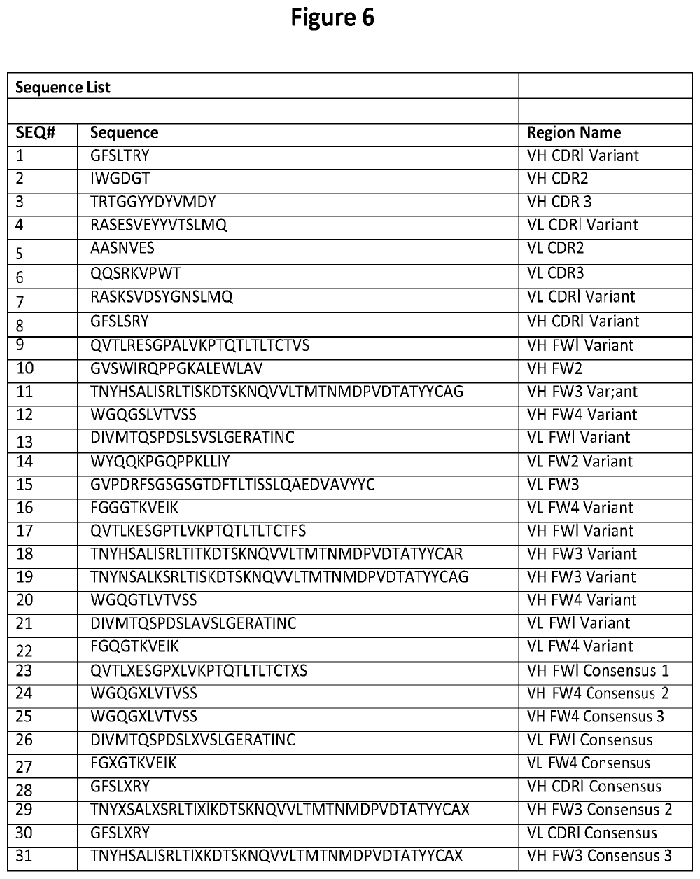

### DETAILED DESCRIPTION OF THE INVENTION

Antibodies

Antibodies and fragments of the antibodies as provided herein may have a variable heavy (VH) domain with complementarity determining regions (CDRs) 1, 2, and 3; and a variable light (VL) domain having CDRs 1, 2, and 3, where the VH CDRs 1, 2, 3 and the VL CDRs 1, 2, 3 are the same as those described for murine antibody, VAC69. If the antibodies have the same six CDRs as the murine antibody, the antibodies will have a VH CDR1 having the sequence shown in SEQ ID NO:1, a VH CDR2 having the sequence shown in SEQ ID NO:2, a VH CDR3 having the sequence shown in SEQ ID NO:3, a VL CDR1 having the sequence shown in SEQ ID NO:4, a VL CDR2 having the sequence shown in SEQ ID NO:5, and a VL CDR3 having the sequence shown in SEQ ID NO:6. The humanized antibody, while having the same 6 CDRs as that of the murine antibody, will have different framework (FW) sequences. The framework sequences of the humanized antibody may include a VH FW1 having the sequence shown in SEQ ID NO:23, a VH FW2 having the sequence shown in SEQ ID NO:10, a VH FW3 having the sequence shown in SEQ ID NO:24, and a VH FW4 having the sequence shown in SEQ ID NO:25, a VL FW1 having the sequence shown in SEQ ID NO:26, a VL FW2 having the sequence shown in SEQ ID NO:14, a VL FW3 having the sequence shown in SEQ ID NO:15, and a VL FW4 having the sequence shown in SEQ ID NO:27.

An antibody or a fragment of an antibody having the same the six CDRs as the murine VAC69 antibody, i.e., where the VH CDR1 has the sequence shown in SEQ ID NO:1, the VH CDR2 has the sequence shown in SEQ ID NO:2, the VH CDR3 has the sequence shown in SEQ ID NO:3, the VL CDR1 has the sequence shown in SEQ ID NO:4, the VL CDR2 has the sequence shown in SEQ ID NO:5, and the VL CDR3 has the sequence shown in SEQ ID NO:6, may alternatively have a VH FW1 having the sequence shown in SEQ ID NO:9, a VH FW2 having the sequence shown in SEQ ID NO:10, a VH FW3 having the sequence shown in SEQ ID NO:11, a VH FW4 having the sequence shown in SEQ ID NO:12, a VL FW1 having the sequence shown in SEQ ID NO:26, a VL FW2 having the sequence shown in SEQ ID NO:14, a VL FW3 having the sequence shown in SEQ ID NO:15, and a VL FW4 having the sequence shown in SEQ ID NO:27.

An antibody or a fragment of an antibody having the same the six CDRs as the murine VAC69 antibody, i.e., where the VH CDR1 has the sequence shown in SEQ ID NO:1, the VH CDR2 has the sequence shown in SEQ ID NO:2, the VH CDR3 has the sequence shown in SEQ ID NO:3, the VL CDR1 has the sequence shown in SEQ ID NO:4, the VL CDR2 has the sequence shown in SEQ ID NO:5, and the VL CDR3 has the sequence shown in SEQ ID NO:6, may alternatively have a VH FW1 having the sequence shown in SEQ ID NO:17, a VH FW2 having the sequence shown in SEQ ID NO:10, a VH FW3 having the sequence shown in SEQ ID NO:18, a VH FW4 having the sequence shown in SEQ ID NO:20, a VL FW1 having the sequence shown in SEQ ID NO:26, a VL FW2 having the sequence shown in SEQ ID NO:14, a VL FW3 having the sequence shown in SEQ ID NO:15, and a VL FW4 having the sequence shown in SEQ ID NO:27.

A further antibody or antibody fragment having the same six CDRs as the murine VAC69 antibody, i.e., where the VH CDR1 has the sequence shown in SEQ ID NO:1, the VH CDR2 has the sequence shown in SEQ ID NO:2, the VH CDR3 has the sequence shown in SEQ ID NO:3, the VL CDR1 has the sequence shown in SEQ ID NO:4, the VL CDR2 has the sequence shown in SEQ ID NO:5, and the VL CDR3 has the sequence shown in SEQ ID NO:6, may alternatively have a VH FW1 having the sequence shown in SEQ ID NO:23, a VH FW2 having the sequence shown in SEQ ID NO:10, a VH FW3 having the sequence shown in SEQ ID NO:24, a VH FW4 having the sequence shown in SEQ ID NO:25, a VL FW1 having the sequence shown in SEQ ID NO:13, a VL FW2 having the sequence shown in SEQ ID NO:14, a VL FW3 having the sequence shown in SEQ ID NO:15, and a VL FW4 having the sequence shown in SEQ ID NO:16.

An antibody or antibody fragment having the same six CDRs as the murine antibody, i.e., where the VH CDR1 has the sequence shown in SEQ ID NO:1, the VH CDR2 has the sequence shown in SEQ ID NO:2, the VH CDR3 has the sequence shown in SEQ ID NO:3, the VL CDR1 has the sequence shown in SEQ ID NO:4, the VL CDR2 has the sequence shown in SEQ ID NO:5, and the VL CDR3 has the sequence shown in SEQ ID NO:6, may alternatively have a VH FW1 having the sequence shown in SEQ ID NO:23, a VH FW2 having the sequence shown in SEQ ID NO:10, a VH FW3 having the sequence shown in SEQ ID NO:24, a VH FW4 having the sequence shown in SEQ ID NO:25, a VL FW1 having the sequence shown in SEQ ID NO:21, a VL FW2 having the sequence shown in SEQ ID NO:14, a VL FW3 having the sequence shown in SEQ ID NO:15, and a VL FW4 having the sequence shown in SEQ ID NO:16.

Another antibody or antibody fragment having the same six CDRs as the murine antibody, i.e., where the VH CDR1 has the sequence shown in SEQ ID NO:1, the VH CDR2 has the sequence shown in SEQ ID NO:2, the VH CDR3 has the sequence shown in SEQ ID NO:3, the VL CDR1 has the sequence shown in SEQ ID NO:4, the VL CDR2 has the sequence shown in SEQ ID NO:5, and the VL CDR3 has the sequence shown in SEQ ID NO:6, may alternatively have a VH FW1 having the sequence shown in SEQ ID NO:23, a VH FW2 having the sequence shown in SEQ ID NO:10, a VH FW3 having the sequence shown in SEQ ID NO:24, a VH FW4 having the sequence shown in SEQ ID NO:25, a VL FW1 having the sequence shown in SEQ ID NO:21, a VL FW2 having the sequence shown in SEQ ID NO:14, a VL FW3 having the sequence shown in SEQ ID NO:15, and a VL FW4 having the sequence shown in SEQ ID NO:22.

Further, specific, framework sequences of antibodies or antibody fragments having the six CDRs of the murine antibody (where the VH CDR1 has the sequence shown in SEQ ID NO:1, the VH CDR2 has the sequence shown in SEQ ID NO:2, the VH CDR3 has the sequence shown in SEQ ID NO:3, the VL CDR1 has the sequence shown in SEQ ID NO:4, the VL CDR2 has the sequence shown in SEQ ID NO:5, and the VL CDR3 has the sequence shown in SEQ ID NO:6) may have a set of VH and VL frameworks that may be any one of a: (i) VH FW1 having the sequence shown in SEQ ID NO:17, VH FW2 having the sequence shown in SEQ ID NO:10, VH FW3 having the sequence shown in SEQ ID NO:18, VH FW4 having the sequence shown in SEQ ID NO:20, VL FW1 having the sequence shown in SEQ ID NO:21, VL FW2 having the sequence shown in SEQ ID NO:14, VL FW3 having the sequence shown in SEQ ID NO:15, and VL FW4 having the sequence shown in SEQ ID NO:22; or (ii) VH FW1 having the sequence shown in SEQ ID NO:17, VH FW2 having the sequence shown in SEQ ID NO:10, VHFW3 having the sequence shown in SEQ ID NO:18, VH FW4 having the sequence shown in SEQ ID NO:20, VL FW1 having the sequence shown in SEQ ID NO:13, VL FW2 having the sequence shown in SEQ ID NO:14, VL FW3 having the sequence shown in SEQ ID NO:15, and VL FW4 having the sequence shown in SEQ ID NO:16; or (iii) VH FW1 having the sequence shown in SEQ ID NO:9, VH FW2 having the sequence shown in SEQ ID NO:10, VH FW3 having the sequence shown in SEQ ID NO:11, VH FW4 having the sequence shown in SEQ ID NO:12, VL FW1 having the sequence shown in SEQ ID NO:21, VL FW2 having the sequence shown in SEQ ID NO:14, VL FW3 having the sequence shown in SEQ ID NO:15, and VL FW4 having the sequence shown in SEQ ID NO:22; or (iv) VH FW1 having the sequence shown in SEQ ID NO:9, VH FW2 having the sequence shown in SEQ ID NO:10, VH FW3 having the sequence shown in SEQ ID NO:11, VH FW4 having the sequence shown in SEQ ID NO:12, VL FW1 having the sequence shown in SEQ ID NO:21, VL FW2 having the sequence shown in SEQ ID NO:14, VL FW3 having the sequence shown in SEQ ID NO:15, and VL FW4 having the sequence shown in SEQ ID NO:22.

The antibodies and antibody fragments need not have the same VH CDRs 1, 2, and 3 and/or VL CDRs 1, 2, and 3 as that of the murine VAC69 antibody. If the antibodies or antibody fragments have CDRs that differ in sequence from that of the murine antibody, the antibodies or antibody fragments may have a VH CDR1 having the sequence shown in SEQ ID NO:28, a VH CDR2 having the sequence shown in SEQ ID NO:2, a VH CDR3 having the sequence shown in SEQ ID NO:3, a VL CDR1 having the sequence shown in SEQ ID NO:30, a VL CDR2 having the sequence shown in SEQ ID NO:5, and a VL CDR3 having the sequence shown in SEQ ID NO:6.

Antibodies and antibody fragments having CDRs that differ in amino acid sequence from the murine VAC69 antibody may have a set of six CDRs, VH CDRs 1, 2, and 3 and VL CDRs 1, 2, and 3, with amino acid sequences that include: (i) VH CDR1 having the sequence shown in SEQ ID NO:28, VH CDR2 having the sequence shown in SEQ ID NO:2, VH CDR3 having the sequence shown in SEQ ID NO:3, VL CDR1 having the sequence shown in SEQ ID NO:7, VL CDR2 having the sequence shown in SEQ ID NO:5, and VL CDR3 having the sequence shown in SEQ ID NO:6; or (ii) VH CDR1 having the sequence shown in SEQ ID NO:8, VH CDR2 having the sequence shown in SEQ ID NO:2, VH CDR3 having the sequence shown in SEQ ID NO:3, VL CDR1 having the sequence shown in SEQ ID NO:4, VL CDR2 having the sequence shown in SEQ ID NO:5, and VL CDR3 having the sequence shown in SEQ ID NO:6; or (iii) VH CDR1 having the sequence shown in SEQ ID NO:1, VH CDR2 having the sequence shown in SEQ ID NO:2, VH CDR3 having the sequence shown in SEQ ID NO:3, VL CDR1 having the sequence shown in SEQ ID NO:7, VL CDR2 having the sequence shown in SEQ ID NO:5, and VL CDR3 having the sequence shown in SEQ ID NO:6; or (iv) VH CDR1 having the sequence shown in SEQ ID NO:8, VH CDR2 having the sequence shown in SEQ ID NO:2, VH CDR3 having the sequence shown in SEQ ID NO:3, VL CDR1 having the sequence shown in SEQ ID NO:7, VL CDR2 having the sequence shown in SEQ ID NO:5, and VL CDR3 having the sequence shown in SEQ ID NO:6.

If the antibodies or antibody fragments have a set of VH and VL CDRs where the VH CDR1 has the sequence shown in SEQ ID NO:28, the VH CDR2 has the sequence shown in SEQ ID NO:2, the VH CDR3 has the sequence shown in SEQ ID NO:3, the VL CDR1 has the sequence shown in SEQ ID NO:30, the VL CDR2 has the sequence shown in SEQ ID NO:5, and the VL CDR3 has the sequence shown in SEQ ID NO:6; then the antibodies or antibody fragments may have VH and VL domain frameworks where the VH FW1 has the sequence shown in SEQ ID NO:23, the VH FW2 has the sequence shown in SEQ ID NO:10, the VH FW3 has the sequence shown in SEQ ID NO:29, the VH FW4 has the sequence shown in SEQ ID NO:25, the VL FW1 has the sequence shown in SEQ ID NO:26, the VL FW2 has the sequence shown in SEQ ID NO:14, the VL FW3 has the sequence shown in SEQ ID NO:15, and the VL FW4 has the sequence shown in SEQ ID NO:27.

If the antibodies or antibody fragments have CDRs in which the: (i) VH CDR1 has the sequence shown in SEQ ID NO:28, VH CDR2 has the sequence shown in SEQ ID NO:2, VH CDR3 has the sequence shown in SEQ ID NO:3, VL CDR1 has the sequence shown in SEQ ID NO:7, VL CDR2 has the sequence shown in SEQ ID NO:5, and VL CDR3 has the sequence shown in SEQ ID NO:6; or the (ii) VH CDR1 has the sequence shown in SEQ ID NO:8, VH CDR2 has the sequence shown in SEQ ID NO:2, VH CDR3 has the sequence shown in SEQ ID NO:3, VL CDR1 has the sequence shown in SEQ ID NO:4, VL CDR2 has the sequence shown in SEQ ID NO:5, and VL CDR3 has the sequence shown in SEQ ID NO:6; or the (iii) VH CDR1 has the sequence shown in SEQ ID NO:1, VH CDR2 has the sequence shown in SEQ ID NO:2, VH CDR3 has the sequence shown in SEQ ID NO:3, VL CDR1 has the sequence shown in SEQ ID NO:7, VL CDR2 has the sequence shown in SEQ ID NO:5, and VL CDR3 has the sequence shown in SEQ ID NO:6; or the (iv) VH CDR1 has the sequence shown in SEQ ID NO:8, VH CDR2 has the sequence shown in SEQ ID NO:2, VH CDR3 has the sequence shown in SEQ ID NO:3, VL CDR1 has the sequence shown in SEQ ID NO:7, VL CDR2 has the sequence shown in SEQ ID NO:5, and VL CDR3 has the sequence shown in SEQ ID NO:6; then the antibodies or antibody fragments may further have VH and VL domain framework regions where the VH FW1 has the sequence shown in SEQ ID NO:23, the VH FW2 has the sequence shown in SEQ ID NO:10, the VH FW3 has the sequence shown in SEQ ID NO:29, the VH FW4 has the sequence shown in SEQ ID NO:25, the VL FW1 has the sequence shown in SEQ ID NO:26, the VL FW2 has the sequence shown in SEQ ID NO:14, the VL FW3 has the sequence shown in SEQ ID NO:15, and the VL FW4 has the sequence shown in SEQ ID NO:27.

If the antibody or antibody fragment has the set of VH and VL CDRs wherein the VH CDR1 has the sequence shown in SEQ ID NO:28, the VH CDR2 has the sequence shown in SEQ ID NO:2, the VH CDR3 has the sequence shown in SEQ ID NO:3, the VL CDR1 has the sequence shown in SEQ ID NO:7, the VL CDR2 having the sequence shown in SEQ ID NO:5, and the VL CDR3 has the sequence shown in SEQ ID NO:6; then the antibody or antibody fragment may have a set of VH and VL frameworks where the VH FW1 has the sequence shown in SEQ ID NO:23, the VH FW2 has the sequence shown in SEQ ID NO:10, the VH FW3 has the sequence shown in SEQ ID NO:29, the VH FW4 has the sequence shown in SEQ ID NO:25, the VL FW1 has the sequence shown in SEQ ID NO:13, the VL FW2 has the sequence shown in SEQ ID NO:14, the VL FW3 has the sequence shown in SEQ ID NO:15, and the VL FW4 has the sequence shown in SEQ ID NO:16.

If the antibody or antibody fragment has the set of VH and VL CDRs wherein the VH CDR1 has the sequence shown in SEQ ID NO:8, the VH CDR2 has the sequence shown in SEQ ID NO:2, the VH CDR3 has the sequence shown in SEQ ID NO:3, the VL CDR1 has the sequence shown in SEQ ID NO:4, the VL CDR2 has the sequence shown in SEQ ID NO:5, and the VL CDR3 has the sequence shown in SEQ ID NO:6; then the antibody or antibody fragment may have VH and VL frameworks where the VH FW1 has the sequence shown in SEQ ID NO:9, the VH FW2 has the sequence shown in SEQ ID NO:10, the VH FW3 has the sequence shown in SEQ ID NO:19, the VH FW4 has the sequence shown in SEQ ID NO:12, the VL FW1 has the sequence shown in SEQ ID NO:26, the VL FW2 has the sequence shown in SEQ ID NO:14, the VL FW3 has the sequence shown in SEQ ID NO:15, and the VL FW4 has the sequence shown in SEQ ID NO:27.

If the antibody or antibody fragment has a set of VH and VL CDRs wherein the VH CDR1 has the sequence shown in SEQ ID NO:1, the VH CDR2 has the sequence shown in SEQ ID NO:2, the VH CDR3 has the sequence shown in SEQ ID NO:3, the VL CDR1 has the sequence shown in SEQ ID NO:7, the VL CDR2 has the sequence shown in SEQ ID NO:5, and the VL CDR3 has the sequence shown in SEQ ID NO:6; then the antibody may have a set of VH and VL frameworks where the VH FW1 has the sequence shown in SEQ ID NO:23, the VH FW2 has the sequence shown in SEQ ID NO:10, the VH FW3 has the sequence shown in SEQ ID NO:31, the VH FW4 has the sequence shown in SEQ ID NO:25, the VL FW1 has the sequence shown in SEQ ID NO:13, the VL FW2 has the sequence shown in SEQ ID NO:14, the VL FW3 has the sequence shown in SEQ ID NO:15, and the VL FW4 has the sequence shown in SEQ ID NO:16.

If the antibodies or antibody fragments have CDRs that differ in amino acid sequence from those of the VAC69 murine antibody, then the antibodies or antibody fragments may have a VH domain and a VL domain that include any of the sets of the following sequences: (i) a VH FW1 having the sequence shown in SEQ ID NO:9, a VH CDR1 having the sequence shown in SEQ ID NO:1, a VH FW2 having the sequence shown in SEQ ID NO:10, a VH CDR2 having the sequence shown in SEQ ID NO:2, a VH FW3 having the sequence shown in SEQ ID NO:11, a VH CDR3 having the sequence shown in SEQ ID NO:3, a VH FW4 having the sequence shown in SEQ ID NO:12, a VL FW1 having the sequence shown in SEQ ID NO:13, a VL CDR1 having the sequence shown in SEQ ID NO:7, a VL FW2 having the sequence shown in SEQ ID NO:14, a VL CDR2 having the sequence shown in SEQ ID NO:5, a VL FW3 having the sequence shown in SEQ ID NO:15, a VL CDR3 having the sequence shown in SEQ ID NO:6, and a VL FW4 having the sequence shown in SEQ ID NO:16; or (ii) a VH FW1 having the sequence shown in SEQ ID NO:9, a VH CDR1 having the sequence shown in SEQ ID NO:8, a VH FW2 having the sequence shown in SEQ ID NO:10, a VH CDR2 having the sequence shown in SEQ ID NO:2, a VH FW3 having the sequence shown in SEQ ID NO:19, a VH CDR3 having the sequence shown in SEQ ID NO:3, a VH FW4 having the sequence shown in SEQ ID NO:12, a VL FW1 having the sequence shown in SEQ ID NO:13, a VL CDR1 having the sequence shown in SEQ ID NO:7, a VL FW2 having the sequence shown in SEQ ID NO:14, a VL CDR2 having the sequence shown in SEQ ID NO:5, a VL FW3 having the sequence shown in SEQ ID NO:15, a VL CDR3 having the sequence shown in SEQ ID NO:6, and a VL FW4 having the sequence shown in SEQ ID NO:16; or (iii) a VH FW1 having the sequence shown in SEQ ID NO:17, a VH CDR1 having the sequence shown in SEQ ID NO:1, a VH FW2 having the sequence shown in SEQ ID NO:10, a VH CDR2 having the sequence shown in SEQ ID NO:2, a VH FW3 having the sequence shown in SEQ ID NO:18, a VH CDR3 having the sequence shown in SEQ ID NO:3, a VH FW4 having the sequence shown in SEQ ID NO:20, a VL FW1 having the sequence shown in SEQ ID NO:13, a VL CDR1 having the sequence shown in SEQ ID NO:7, a VL FW2 having the sequence shown in SEQ ID NO:14, a VL CDR2 having the sequence shown in SEQ ID NO:5, a VL FW3 having the sequence shown in SEQ ID NO:15, a VL CDR3 having the sequence shown in SEQ ID NO:6, and a VL FW4 having the sequence shown in SEQ ID NO:16; or (iv) a VH FW1 having the sequence shown in SEQ ID NO:9, a VH CDR1 having the sequence shown in SEQ ID NO:8, a VH FW2 having the sequence shown in SEQ ID NO:10, a VH CDR2 having the sequence shown in SEQ ID NO:2, a VH FW3 having the sequence shown in SEQ ID NO:19, a VH CDR3 having the sequence shown in SEQ ID NO:3, a VH FW4 having the sequence shown in SEQ ID NO:12, a VL FW1 having the sequence shown in SEQ ID NO:13, a VL CDR1 having the sequence shown in SEQ ID NO:4, a VL FW2 having the sequence shown in SEQ ID NO:14, a VL CDR2 having the sequence shown in SEQ ID NO:5, a VL FW3 having the sequence shown in SEQ ID NO:15, a VL CDR3 having the sequence shown in SEQ ID NO:6, and a VL FW4 having the sequence shown in SEQ ID NO:16; or (v) a VH FW1 having the sequence shown in SEQ ID NO:9, a VH CDR1 having the sequence shown in SEQ ID NO:8, a VH FW2 having the sequence shown in SEQ ID NO:10, a VH CDR2 having the sequence shown in SEQ ID NO:2, a VH FW3 having the sequence shown in SEQ ID NO:19, a VH CDR3 having the sequence shown in SEQ ID NO:3, a VH FW4 having the sequence shown in SEQ ID NO:12, a VL FW1 having the sequence shown in SEQ ID NO:21, a VL CDR1 having the sequence shown in SEQ ID NO:4, a VL FW2 having the sequence shown in SEQ ID NO:14, a VL CDR2 having the sequence shown in SEQ ID NO:5, a VL FW3 having the sequence shown in SEQ ID NO:15, a VL CDR3 having the sequence shown in SEQ ID NO:6, and a VL FW4 having the sequence shown in SEQ ID NO:22.

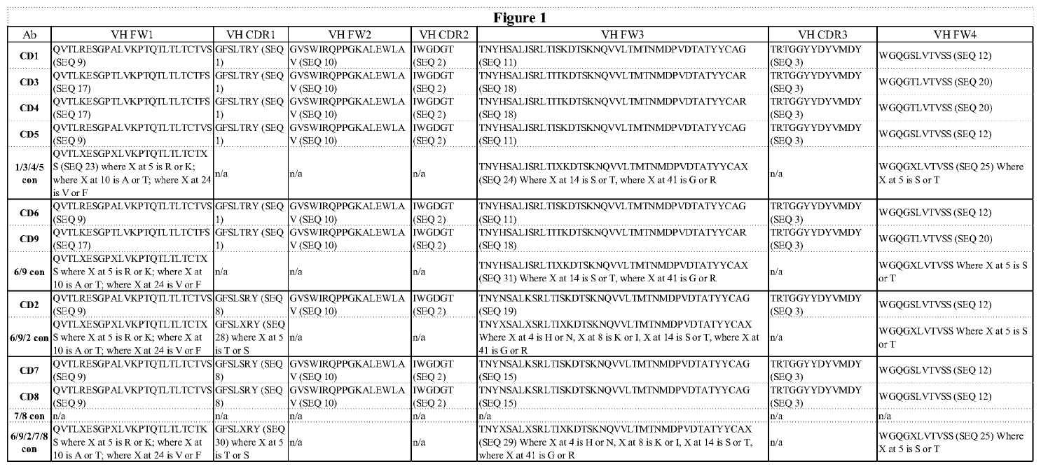

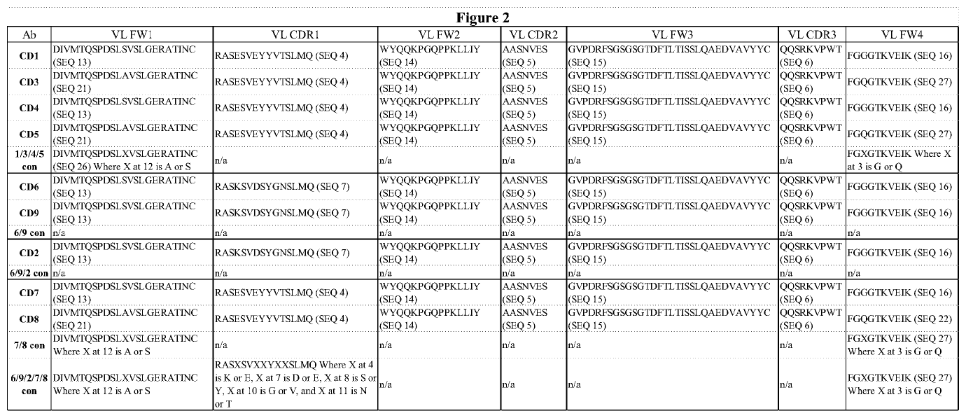

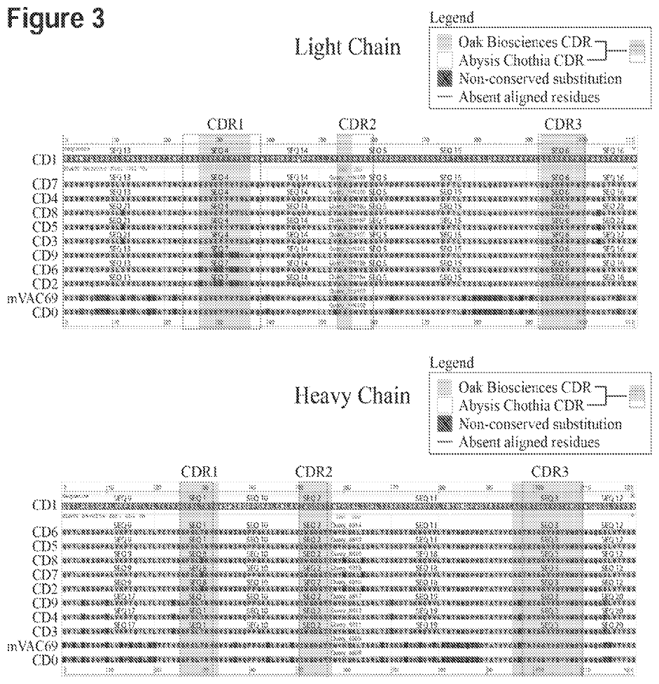

While all the sequences noted as CDR and FW sequences for the antibodies and antibody fragments have been described based on the Absys Chothia method (as shown in FIG. 3), the CDR and FW sequences of the Absys Chothia method may readily be substituted for those as determined by the Oak Biosciences method (FIG. 3). The antibodies or antibody fragments may have a VH domain and a VL domain having any sequence or any combination of sequences as shown in FIGS. 1-3.

The antibody or antibody fragment may be of an antibody of any isotype or a fragment of an antibody of any isotype. For example, it may be an IgG, an IgA, an IgM, an IgE, or an IgD antibody. It may be a fragment of an IgG, IgA, IgM, IgE, or an IgD antibody. A fragment of the antibody may be a Fab, a F(ab′)², a Fab′, a Fv, a scFv, a Fd′, a Fab², or a Fd, or any other as known in the art.

The antibodies or antibody fragments may be included in a composition. The composition may be sterile. An example of such a composition may therefore be a sterile composition containing an antibody or a fragment of an antibody as described herein. It may be a sterile composition that includes combination of antibodies or antibody fragments wherein one or the one or more antibodies or antibody fragments are an antibody or antibody fragment as described herein. The sterile composition may be suitable for use as a pharmaceutical composition. A pharmaceutical composition may be formulated for intravenous, intramuscular, subcutaneous, or intraperitoneal administration. The pharmaceutical composition may further contain an excipient, carrier, diluent, or stabilizer. Those of skill in the art are aware of suitable excipients, carriers, diluents, or stabilizers that may be employed to formulate a pharmaceutical composition capable of being administered to a patient.

The antibodies or antibody fragments may be included in a composition for use as a reagent, e.g., as a research tool. Such compositions may or may not be sterile compositions. Such compositions may contain, in addition to any of the antibodies or antibody fragments described herein, a cryoprotectant such as glycerol or ethylene glycol (if the antibody composition is to be stored at a below-freezing temperature), an antimicrobial agent such as sodium azide, a protease inhibitor, an antioxidant such as beta-mercaptoethanol or dithiothreitol, a carrier protein such as bovine serum albumin, a metal chelator such as ethylenediaminetetraacetic acid, an emulsifier such as polysorbate 20, or a buffer such as phosphate buffered saline. Compositions may contain any of these reagents, other additional reagents, or any combination of these or other reagents. One of skill in the art is aware of how to combine reagents in a composition containing an antibody or antibody fragment, as well as appropriate concentrations of the reagents present in such a composition.

The antibodies and antibody fragments described herein may be in a solid composition, e.g., lyophilized antibody composition.

The antibodies and antibody fragments may further include a detectable label. The antibodies and antibody fragments may be labeled with any molecule, as appropriate, understood by those of skill in the art. Appropriate labels may be selected based on the intended use of the antibody or antibody fragment. The antibodies may be labeled with enzymes such as horse radish peroxidase, alkaline phosphatase, horseradish galactosidase, or luciferase, if they are to be used in a detection assay, e.g., enzyme-linked immunosorbent assay. The enzymes may be labeled with fluorescent dyes if they are going to be used in an immunofluorescence assay, or may be labeled with fluorescent proteins or dyes if they are going to be used in a flow cytometry assay. Fluorescent dyes that may be used to label the antibody may, for example, include FLAG, GFP, YRF, RFP, Cy3, Cy5, Texas Red, rhodamine, Alexa fluors. The detectable label may further be a radioisotope such as ³H, ¹⁴C, ¹⁵N, ³⁵S, ⁹⁰Y, ¹²⁵I, or ¹³¹I if the antibody or antibody fragment is to be used in a, e.g., therapeutic protocol.

The antibody or antibody fragment may further include, or may be conjugated to, an antitumor drug. The antitumor drug that may be useful for treating cancer or a tumor. Examples of these antitumor drugs include an angiogenesis inhibitor, a DNA damaging agent such as calicheamicin, an anthracycline, a duocarmycin, or a pyrrolobenzodiazepine, or microtubule inhibitor such as an auristatin or maytansine.

The antibody or antibody fragment may further include, or may be conjugated to, a biological response modifier. A biological response modifier may modify a patient's immune response to myeloma or ovarian cancer cells. Immune response modifiers include lymphokines, such as tumor necrosis factor, interleukins, lymphotoxin, macrophage activating factor (MAF), migration inhibition factor (MIF), colony stimulating factor (CSF), and interferon.

The antibodies and antibody fragments described herein may be in a kit. The kit may further contain instructions for using the antibodies or antibody fragments. The instructions may relate to use of the antibody in an in vitro or in vivo detection assay or detection procedure. The instructions may relate to the use of the antibody in a therapeutic protocol. The instructions may relate to final preparation, e.g., dilution or dissolving, of the antibody for formulation and/or readying for use in an in vitro detection assay, an in vivo detection assay, or in a therapeutic protocol. The kit may further contain one or more aqueous solutions and may yet further contain additional agents, e.g., therapeutic agent or agents, in one or more separate containers.

Polynucleotides, Vectors, and Cells

Polynucleotides encoding the VH domain and/or VL domain of the antibodies or antibody fragments described herein are also provided. The polynucleotides may be DNA, or may be RNA or may be modified DNA or RNA, e.g., PNA.

One of skill in the art could readily produce nucleic acid sequences that encode the VH and VL domains of the antibodies with the knowledge of the amino acid sequences of the VH and VL domains as provided herein. See FIGS. 1-3. The polynucleotides may include the sequence of both the VH domain and the VL domain, or of only the VH domain, or of only the VL domain of the antibody or antibody fragment.

The polynucleotides may be included in a vector. The vector may be an expression vector. The expression vector may be selected based on whether the antibody will be expressed in a transient transfection system or whether the antibody will be integrated into a host cell chromosome. The expression vector may also be selected based on the cell type, e.g., mammalian, bacterial, plant, or fungi, that will express the antibody. Expression vectors for cells may be purchased from a vendor as many are commercially available, or may be proprietary or may be prepared by one of skill in the art.

An expression vector may have a strong promoter such as the immediate early cytomegalovirus promotor, cellular elongation factor (EF) I-alpha promoter. It may have polyadenylation sites from the simian virus (SV) 40 or from bovine growth hormone for improved mRNA stability and translation efficiency. It may also have selection markers, cloning sites and other regulatory sequences, e.g., an internal ribosomal entry site.

A host cell may contain the vector. The host cell may be a mammalian cell such as a Chinese Hamster Ovary cells, a human retinal cell from line Per.C6, NS0, a baby hamster kidney cell, HKB11 cell, MCF-7 cell, HeLa cell, COS-7 cell, VERO cell, 293 cell, CV1 cell, or human embryonic kidney cell HEK293. The host cell may be an insect cell such as a cell of the insect cell line Sf-9 or Sf-21 of Spodoptera frugiperda. The host cell may be a plant cell in a transgenic plant such as Nicotiana tabacum, Nicotiana benthamiana, Nicotiana tabacum, or Nicotiana benthamiana.

The host cell may express the antibody or antibody fragment. The antibody or antibody may be isolated, and/or purified following expression by the host cell.

Detecting Ovarian Cancer Cells or Myeloma Cells

The antibodies and/or fragments of antibodies described herein can be used in methods of detecting cancer cells in a patient. In such methods, a patient sample is contacted with an antibody or antibody fragment as described herein. The presence or absence of binding between the antibody or antibody fragment and cancer cells in the patient sample is detected. Presence of cancer is detected if there is detection of binding of the antibody or antibody fragment to cancer cells. Absence of cancer cells is determined if there is not detection of binding of the antibody or antibody fragment. The cancer cells may be ovarian cancer cells or myeloma cells.

In the method of detecting cancer cells the patient may be a patient suspected of having cancer, or a patient undergoing routine screening as part of a routine well visit examination. The patient may also be known to have cancer and be about to undergo treatment or be a patient who is in the process of being treated for cancer. If the patient is known to have cancer, the patient may also be being monitored for progression of cancer or may be getting screened for the purposes of selecting an appropriate therapeutic treatment regimen for the cancer.

In the method a patient sample is contacted with an antibody or fragment of an antibody as described herein. The patient sample may be a tissue sample or a fluid sample. If the sample is a fluid sample it may be a blood sample, serum sample, or a urine sample. If the sample is a tissue sample it may be from a biopsy, such as bone marrow sample in the case of myeloma, or an ovarian tissue sample as in the case of ovarian cancer.

The patient sample may be obtained from the patient and tested at the same facility, e.g., hospital, clinic, or office, from which it was obtained. The patient sample may be obtained from the patent and sent to a second facility for testing. The second facility will obtain the sample from the first facility. Alternatively, the patient sample may be the patient, i.e., in vivo testing, who will be imaged to determine antibody binding to cancer cells, and possibly to further determine localization of the cancer cells in the patient.

The antibody or antibody fragment, depending on the assay type for detecting the cancer cells, may be labeled. Labels, discussed above, may include enzymes, fluorescent proteins or dyes, or radionuclides. The antibodies may be labeled with enzymes such as horse radish peroxidase, alkaline phosphatase, horseradish galactosidase, or luciferase. If the antibody is labeled with a fluorescent dye it may be labeled with cyan fluorescent protein, green fluorescent protein, yellow fluorescent protein, red fluorescent protein, Cy3, Cy5, Texas Red, rhodamine, or Alexa fluors. If the antibody or antibody is labeled with a radioisotope it may be labeled with ³H, ¹⁴C, ¹⁵N, ³⁵S, ⁹⁰Y, ¹²⁵I, or ¹³¹I. Those of skill in the art are aware of appropriate labels for antibodies and how to label antibodies. Methods for detection utilizing labeled antibodies include patient imaging by administering the antibody to a patient, or ELISA, flow cytometry or immunohistochemistry.

Diagnosing Myeloma or Ovarian Cancer

In a method of diagnosing cancer, a patient sample is contacted with an antibody or a fragment of an antibody as described herein. The presence or absence of binding between the antibody or antibody fragment and cancer cells is detected. The patient is diagnosed with cancer if binding is detected. The cancer may be myeloma or ovarian cancer. The cancer may be fallopian tube cancer or peritoneal cancer.

In such a diagnosis method, the patient may be a patient suspected of having cancer, or a patient undergoing routine screening as part of a routine well visit examination. The patient may submit the patient sample, if it is a part of a routine well visit examination, where the patient sample is a fluid sample, e.g., blood sample, serum sample, or urine sample. The patient may submit the sample, if the patient is suspected of having cancer as either a fluid sample such as a blood sample, serum sample, or urine sample, or may submit a biopsy sample such as a bone marrow sample in the case of myeloma, or an ovarian tissue sample as in the case of ovarian cancer. If the patient is suspected of having cancer, the patient may the patient sample, i.e., in vivo testing, and the patient may be imaged to determine antibody binding to cancer cells in the patient.

If the method of diagnosing is performed on a patient sample, where the method is performed in vitro, the sample may be obtained from the patient, by removing an appropriate sample from the body of the patient and/or may be obtained from the patient by its receipt at an appropriate facility that will contact the sample with the antibody or antibody fragment to detect the presence or absence of binding of the antibody or antibody fragment with cells in the sample.

The antibody or antibody fragment that binds to cancer cells, if cancer cells are present in the sample, may be labeled to ease the detection binding of the antibody or antibody fragment to cells in the sample. The antibody or antibody fragment may be labeled with any suitable material including enzymes, fluorescent proteins or dyes, or radionuclides. The antibodies may be labeled with enzymes such as horse-radish peroxidase, alkaline phosphatase, horseradish galactosidase, or luciferase. If the antibody is labeled with a fluorescent dye it may be labeled with blue fluorescent protein, green fluorescent protein, yellow fluorescent protein, red fluorescent protein, Cy3, Cy5, Texas Red, rhodamine, or Alexa fluors. If the antibody or antibody is labeled with a radioisotope it may be labeled with ³H, ¹⁴C, ¹⁵N, ³⁵S, ⁹⁰Y, ¹²⁵I, or ¹³¹I. Those of skill in the art are aware of appropriate labels for antibodies and how to label antibodies, based on the method to be employed in any given method of detection. Methods for detection include patient imaging by administering the antibody to a patient, and ELISA, flow cytometry and immunohistochemistry.

In the diagnosis methods, diagnosis may include diagnosing the patient with the cancer and may further include an indication of stage of cancer and or anticipated prognoses of the cancer.

Methods of Treating a Patient with Myeloma or Ovarian Cancer

In a method for treating a patient with cancer, an effective amount of any of the antibodies or antibody fragments described herein are administered to a cancer patient. The cancer patient may be a myeloma or an ovarian cancer patient. The patient may be fallopian tube cancer or peritoneal cancer patient.

An effective amount of antibody may be any that reduces the burden of cancer cells in the patient, slows the progression of the cancer in the patient, increases chances of survival of the patient, increases survival time for the patient, or alleviates symptoms of cancer in the patient.

The antibody or fragment of the antibody that treats the patient may be administered intravenously, intraperitoneally, intramuscularly, subcutaneously, intracavity, or transdermally. Administration of the antibody or fragment of the antibody will be as a pharmaceutical composition, and as a composition that is sterile. If the antibody or antibody fragment is administered parenterally it may be administered as a sterile aqueous or non-aqueous solution, suspension, or emulsion. Examples of non-aqueous solvents are propylene glycol, polyethylene glycol, vegetable oils such as olive oil, and injectable organic esters such as ethyl oleate. Examples of aqueous carriers include water, alcoholic/aqueous solutions, emulsions or suspensions, including saline and buffered media. If the antibody or antibody fragment is administered intravenously, examples of intravenous vehicles include fluid and nutrient replenishers, and electrolyte replenishers (such as those based on Ringer's dextrose). Preservatives and other additives may also be present such as, for example, antimicrobials, anti-oxidants, chelating agents, and/or inert gases.

The antibody or fragment of the antibody that treats the patient may further include a therapeutic moiety. A therapeutic moiety could be any that enhances the ability of the antibody or antibody fragment to kill or inhibit growth of cancer cells. The therapeutic moiety may be, for example, a peptide, a small molecule, or a radioisotope. If the therapeutic moiety is a peptide, the therapeutic moiety may be a toxin such as diphtheria toxin or modified diphtheria toxin or ricin or modified ricin. If the therapeutic moiety is a small molecule or drug it may be doxorubicin, an auristatin, a maytansine, or a calicheamicin. If the therapeutic moiety is a ⁹⁰Y, ¹²⁵I or ¹³¹I.

The antibody or fragment of the antibody that treats the patient may be administered according to any appropriate schedule and at any appropriate dose. Appropriate doses may be in the range of 0.1 mg/kg to 500 mg/kg, and may be 0.1 mg/kg or 1 mg/kg, or 10 mg/kg, or 50 mg/kg, or 100 mg/kg or 200 mg/kg, or 250 mg/kg or 500 mg/kg. The dosing schedule may be once a week, once a month, once every three months or once every six months.

Administration of the antibody or antibody fragment may be in combination with one or more other therapeutic agents. The therapeutic agent or agents administered in combination with the antibody or antibody fragments may be administered at the same time and in a single delivery, or may be at the same time but in different deliveries, or may be at different times, e.g., different days or different times on the same day, in the course of a treatment regimen.

Therapeutic agents that may be combined with the antibody or antibody fragment to treat a patient with cancer may be any that further improves efficacy of the antibody or antibody in the treatment. Therapeutic agents may not only be those that kill or inhibit growth of cancer cells. The therapeutic agents may also alleviate symptoms of the cancer or symptoms of side effects of therapeutic agents employed to treat the cancer.

If the cancer is myeloma, the therapeutic agent or agents that may be administered with the antibody or antibody fragments disclosed herein may further treat the cancer and may be daratumumab, doxorubicin, elotuzumab, panobinostat, carflizomib, ixazomib, pomalidomide, lenalidomide, thalidomide, bortezomib, plitidepsin, NPI-0052, bendamustine, or vorinostat or combinations thereof. If the cancer is myeloma, the therapeutic agent or agents that may be administered with the antibody or antibody fragments disclosed herein may be a supportive agent such as bisphosphonates for bone strength, growth factors such as erythropoietin or colony stimulating factors, stem cell transplants to restore hematopoietic stem cells, or supplements high in iron folate or vitamin B-12 for anemia. A further therapy, not an agent, may be administered such as radiation.

If the cancer is ovarian cancer, the therapeutic agent or agents that may be administered with the antibody or antibody fragments disclosed herein may further treat the cancer and may be one or more of melphalan, bevacizumab, carboplatin, cyclophosphamide, doxorubicin, docetaxel, gemcitabine, topotecan, olaparib, melphalan, paclitaxel (taxol), oxaliplatin, cisplatin, thiotepa or combinations thereof such as carboplatin-taxol, gemcitabine-cisplatin.

Methods of Killing or Inhibiting Growth of Myeloma or Ovarian Cancer Cells

In a method for killing or inhibiting growth of cancer cells, cancer cells are contacted with an antibody or antibody fragment as described herein. The amount of antibody or antibody fragment contacted with the cancer cells is sufficient to kill or inhibit the growth of the cancer cells. The cancer cells may be myeloma cells or may be ovarian cancer cells. The cancer cells may be fallopian tube cancer cells or peritoneal cancer cells.

The cancer cells in the method may be obtained from a patient or may be of a cancer cell line. If the cancer cells in the method are obtained from a patient, the method may be part of a screening protocol to determine an appropriate dose and/or dosing regimen of the antibody or antibody fragment to be administered to the cancer patient from whom the cancer cells were obtained. It may, alternatively, be part of a screening protocol to determine an appropriate further therapeutic agent to administer to the cancer patient from whom the cancer cells were obtained.

The cancer cells may be killed or may have their growth inhibited in screening assays to identify optimal combinations of therapeutic agents to treat cancers or in preparation for clinical submissions or to conduct basic research to dissect mechanisms of cancer genesis or perpetuation.

The amount of antibody or antibody fragment contacted with the cancer cells that is sufficient to kill or inhibit the growth of the cancer cells can readily be determined by one of skill in the art. Killing of the cancer cells or inhibiting the growth of the cancer cells may be determined by successively increasing the amount of antibody or antibody fragment administered to the cells. Killing the cancer cells may be killing of all cancer cells in the method or may be killing of 95%, 90%, 80%, 75%, 70%, 60%, 50%, 40%, 30%, 25%, 20%, or 10% of the cancer cells in the method. Similarly, inhibiting growth of the cancer cells in the method may be inhibiting growth of all the cancer cells in the method. Inhibiting growth of the cancer cells may be inhibiting growth of 95%, 90%, 80%, 75%, 70%, 60%, 50%, 40%, 30%, 25%, 20%, or 10% of the cancer cells in the method.

The antibody or fragment of the antibody administered to the cancer cells may further include a therapeutic moiety. A therapeutic moiety could be any that enhances the ability of the antibody or antibody fragment to kill or inhibit growth of cancer cells. The therapeutic moiety may be, for example, a peptide, a small molecule, or a radioisotope. If the therapeutic moiety is a peptide, the therapeutic moiety may be a toxin such as diphtheria toxin or modified diphtheria toxin or ricin or modified ricin. If the therapeutic moiety is a small molecule or drug it may be doxorubicin, an auristatin, a maytansine, or a calicheamicin. If the therapeutic moiety is a radioisotope it may be ⁹⁰Y, ¹²⁵I, or ¹³¹I.

The antibody or antibody fragment administered to the cancer cells may be combined with a further therapeutic agent that improves efficacy of the antibody or antibody fragment in killing or inhibiting growth of the cancer cells. The therapeutic agent may be daratumumab, doxorubicin, elotuzumab, panobinostat, carflizomib, ixazomib, pomalidomide, lenalidomide, thalidomide, bortezomib, plitidepsin, NPI-0052, bendamustine, vorinostat, melphalan, bevacizumab, carboplatin, cyclophosphamide, doxorubicin, docetaxel, gemcitabine, topotecan, olaparib, melphalan, paclitaxel (taxol), oxaliplatin, cisplatin, thiotepa and combinations thereof.

The antibody or antibody fragment administered to the cancer cells may be combined with cell signaling moieties to promote cellular responses against the cancer cells. Examples of this include the addition of CD3z, CD28, or 4-1BB intracellular signaling domains to create a chimeric antigen receptor. Wherein the antibody or antibody fragment mediates a cellular signaling response on the cell to effect the cancer cell.

### EXAMPLES

### Example 1—Humanization of VAC69 Murine Antibodies

Humanized antibodies, based on murine antibody VAC69, were produced as potential therapeutic agents for use in humans. The use of humanized antibodies, in place of murine antibodies, would reduce human anti-mouse antibody (HAMA) responses in patients undergoing therapy and would be of even greater importance in prolonged engraftment procedures such as chimeric antigen receptor (CAR)-T cell therapy or CAR-NK cell therapy. Using bioinformatics-based sequence analysis and modeling software, the antibody variable regions were blended to human donor sequences to create a panel of humanized heavy and light chains. The humanized chains were then codon optimized for expression, synthesized, and tested against the parent antibody for relative binding affinity via competition analysis. Sequences and sequence alignments for the antibody variable heavy and variable light chains are provided in FIGS. 1-3.

### Example 2—Humanized VAC69 Antibodies Compete with VAC69 for Antigen Binding

Competitive binding to determine the Ki of the humanized VAC69 variants was performed using a modified version of the flow cytometry protocol developed by Benedict (Benedict et al. J Immunol. Methods. 201(1997)223-31). The protocol used to determine Ki utilized directly-labeled antibody as opposed to Benedict's indirect labeling method.

Prior to the start of the experiment, antigen-expressing tumor cells (U266) were grown in cRPMI culture media. Fluorescently-labeled parental mVAC69 antibody was prepared using the alexa488 fluorescent labeling kit (Thermo #A20181) as per the manufacturer's instructions.

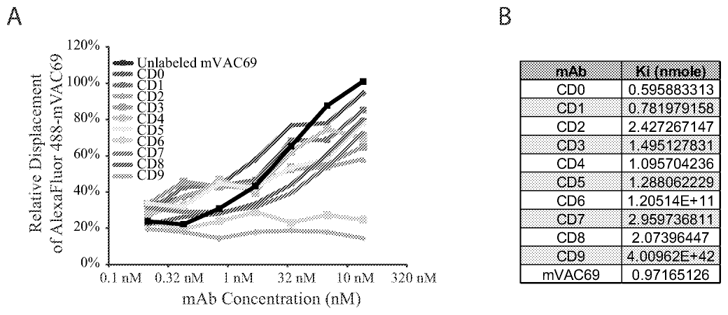

On the day of the experiment, U266 cells were harvested, washed in PBS with 2% FBS/0.1% azide. Unlabeled antibody was added to cells (2E6/mL) for 1 h at 4° C. prior to adding and equal volume of the fluorescently-labeled VAC69 antibody. After an additional 1 hour incubation, cells were washed twice to remove unbound antibody, and fluorescence quantified via flow cytometry. Results of the competitive binding are provided in FIGS. 4A and 4B.

### Example 3—the Humanized Antibodies Exhibit ADCC Activity

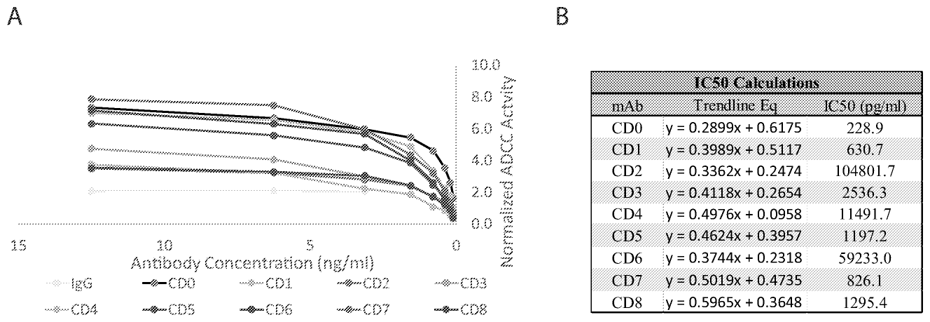

To determine if the humanized antibodies also retained the biological activity of the parental murine antibody, ADCC capacity was next tested. For this experiment, U266 cells were cultured with the Promega ADCC Reporter cell assay system along with titrations of the various humanized antibodies. From these results (FIG. 5), the CD1 humanized antibody was determined to have the highest ADCC activity. The CD1 antibody also had the highest Kd of the humanized antibodies (FIG. 4). Interestingly, the CD4 clone demonstrated only moderate ADCC potential while being the second strongest binding of the constructs.

### CONCLUSIONS

A panel of humanized antibodies was produced from murine antibody VAC69. The humanized antibodies in the panel had one of each of three variable heavy domains matched with one of three variable light chains (See FIGS. 1-3). Subsequent testing, as described in Examples 2 and 3 determined the binding affinity and ADCC capacity for each humanized antibody. This data is summarized below in Table 1. Based on these analyses, the CD1 clone appears to be the closest in affinity and function (ADCC) to the chimeric antibody. There is a loss of affinity between the chimera and the humanized antibody, CD1 (0.59 nM vs. 0.78 nM, respectively). This sub-nanomolar affinity is within range of other clinical-grade antibodies currently available

TABLE 1
Summary Analysis of ADCC and Kd
for Humanized Antibody Clones

| | | CD_{HeavyB} | CD_{HeavyC} |
|---|---|---|---|
| | | (30T−>S, 60H−>N, | (multiple |
| | CD_{HeavyA} | 641−>K) | single) |
| CD_{LightA} | CD1/0.78 | CD7/2.96 | CD4/1.10 |
| CD_{LightB} | CD5/no | CD8/2.07 | CD3/1.50 |
| (125−>A, 104G−>Q) | binding | | |
| CD_{LightC} | CD6/1.29 | CD2/2.42 | CD9/no |
| (CDR1; ESVYYVT −> | | | binding |
| KSVDSYGN) | | | |

Humanized antibody clones and their relative Kd as determined by competitive binding.

We claim:

1. An antibody or an antibody fragment thereof comprising:
a VH domain comprising: a VH FW1, VH CDR1, VH FW2, VH CDR2, VH FW3, VH CDR3, and VHFW4; and
a VL domain comprising a VL FW1, VL CDR1, VL FW2, VL CDR2, VL FW3, VL CDR3, and VL FW4 wherein:
the VH FW1 has the sequence shown in SEQ ID NO:23;
the VH CDR1 has the sequence shown in SEQ ID NO:1;
the VH FW2 has the sequence shown in SEQ ID NO:10;
the VH CDR2 has the sequence shown in SEQ ID NO:2;
the VH FW3 has the sequence shown in SEQ ID NO:24;
the VH CDR3 has the sequence shown in SEQ ID NO:3;
the VH FW4 has the sequence shown in SEQ ID NO:25;
the VL FW1 has the sequence shown in SEQ ID NO:26;
the VL CDR1 has the sequence shown in SEQ ID NO:4;
the VL FW2 has the sequence shown in SEQ ID NO:14;
the VLCDR2 has the sequence shown in SEQ ID NO:5;
the VL FW3 has the sequence shown in SEQ ID NO:15;
the VL CDR3 has the sequence shown in SEQ ID NO:6; and
the VL FW4 has the sequence shown in SEQ ID NO:27.

2. The antibody or antibody fragment of claim 1 wherein:
the VH FW1 has the sequence shown in SEQ ID NO: 9, the VH FW3 has the sequence shown in SEQ ID NO: 11, and the VH FW4 has the sequence shown in SEQ ID NO:12; or
the VH FW1 has the sequence shown in SEQ ID NO: 17, the VH FW3 has the sequence shown in SEQ ID NO: 18, and the VH FW3 has the sequence shown in SEQ ID NO: 20;
or the VL FW1 has the sequence shown in SEQ ID NO: 13 and the VL FW4 has the sequence shown in SEQ ID NO: 16; or
the VL FW1 has the sequence shown in SEQ ID NO:21 and the VL FW4 has the sequence shown in SEQ ID NO: 22; or
the VH FW1 has the sequence shown in SEQ ID NO: 9, the VH FW3 has the sequence shown in SEQ ID NO: 11, the VH FW4 has the sequence shown in SEQ ID NO:12, the VL FW1 has the sequence shown in SEQ ID NO: 13 and the VL FW4 has the sequence shown in SEQ ID NO: 16; or
the VH FW1 has the sequence shown in SEQ ID NO: 17, the VH FW3 has the sequence shown in SEQ ID NO: 18, the VH FW4 has the sequence shown in SEQ ID NO: 20, the VL FW1 has the sequence shown in SEQ ID NO:21 and the VL FW4 has the sequence shown in SEQ ID NO: 22; or
the VH FW1 has the sequence shown in SEQ ID NO: 17, the VH FW3 has the sequence shown in SEQ ID NO: 18, the VH FW4 has the sequence shown in SEQ ID NO: 20, the VL FW1 has the sequence shown in SEQ ID NO: 13 and the VL FW4 has the sequence shown in SEQ ID NO: 16; or
the VH FW1 has the sequence shown in SEQ ID NO: 9, the VH FW3 has the sequence shown in SEQ ID NO: 11, the VH FW4 has the sequence shown in SEQ ID NO:12, the VL FW1 has the sequence shown in SEQ ID NO:21 and the VL FW4 has the sequence shown in SEQ ID NO: 22.

3. An antibody or an antibody fragment thereof comprising a VH domain and a VL domain wherein:
the VH domain comprises a VH CDR1, VH CDR2, and VH CDR3; and the VL domain comprises a VL CDR1, VL CDR2, and VL CDR3; and wherein:
the VH CDR1 has the sequence shown in SEQ ID NO: 28;
the VH CDR2 has the sequence shown in SEQ ID NO: 2;
the VH CDR3 has the sequence shown in SEQ ID NO: 3;
the VL CDR1 has the sequence shown in SEQ ID NO: 30;
the VL CDR2 has the sequence shown in SEQ ID NO:5; and
the VL CDR3 has the sequence shown in SEQ ID NO:6.

4. The antibody or antibody fragment of claim 3 wherein:
the VH CDR1 has the sequence shown in SEQ ID NO: 28;
the VH CDR2 has the sequence shown in SEQ ID NO:2;
the VH CDR3 has the sequence shown in SEQ ID NO: 3;
the VL CDR1 has the sequence shown in SEQ ID NO: 7;
the VL CDR2 has the sequence shown in SEQ ID NO: 5; and
the VL CDR3 has the sequence shown in SEQ ID NO:6.

5. The antibody or antibody fragment of claim 3 wherein:
the VH CDR1 has the sequence shown in SEQ ID NO:8;
the VH CDR2 has the sequence shown in SEQ ID NO:2;
the VH CDR3 has the sequence shown in SEQ ID NO:3;
the VL CDR1 has the sequence shown in SEQ ID NO: 4,
the VL CDR2 has the sequence shown in SEQ ID NO: 5; and
the VL CDR3 has the sequence shown in SEQ ID NO:6

6. The antibody or antibody fragment of claim 4 wherein:
the VH CDR1 has the sequence shown in SEQ ID NO:1;
the VH CDR2 has the sequence shown in SEQ ID NO:2;
the VH CDR3 has the sequence shown in SEQ ID NO:3;
the VL CDR1 has the sequence shown in SEQ ID NO:7;
the VL CDR2 has the sequence shown in SEQ ID NO:5; and
the VL CDR3 has the sequence shown in SEQ ID NO:6.

7. The antibody or antibody fragment of claim 4 wherein:
the VH CDR1 has the sequence shown in SEQ ID NO:8;
the VH CDR2 has the sequence shown in SEQ ID NO:2;
the VH CDR3 has the sequence shown in SEQ ID NO:3;
the VL CDR1 has the sequence shown in SEQ ID NO:7;
the VL CDR2 has the sequence shown in SEQ ID NO:5; and
the VL CDR3 has the sequence shown in SEQ ID NO:6.

8. The antibody or antibody fragment of claim 3 further comprising:
a VH FW1 having the sequence shown in SEQ ID NO: 23;
a VH FW2 having the sequence shown in SEQ ID NO: 10;
a VH FW3 having the sequence shown in SEQ ID NO: 29;
a VH FW4 having the sequence shown in SEQ ID NO: 25;
a VL FW1 having the sequence shown in SEQ ID NO: 26;
a VL FW2 having the sequence shown in SEQ ID NO: 14;
a VL FW3 having the sequence shown in SEQ ID NO: 15; and
a VL FW4 having the sequence shown in SEQ ID NO: 27.

9. The antibody or antibody fragment of claim 4 further comprising:
a VH FW1 having the sequence shown in SEQ ID NO: 23;
a VH FW2 having the sequence shown in SEQ ID NO: 10;
a VH FW3 having the sequence shown in SEQ ID NO: 29;
a VH FW4 having the sequence shown in SEQ ID NO: 25;
a VL FW1 having the sequence shown in SEQ ID NO: 13;
a VL FW2 having the sequence shown in SEQ ID NO: 14;
a VL FW3 having the sequence shown in SEQ ID NO: 15; and
a VL FW4 having the sequence shown in SEQ ID NO: 16.

10. The antibody or antibody fragment of claim 5 further comprising:
a VH FW1 having the sequence shown in SEQ ID NO: 9;
a VH FW2 having the sequence shown in SEQ ID NO: 10;
a VH FW3 having the sequence shown in SEQ ID NO: 19
a VH FW4 having the sequence shown in SEQ ID NO: 12;
a VL FW1 having the sequence shown in SEQ ID NO: 26;
a VL FW2 having the sequence shown in SEQ ID NO: 14;
a VL FW3 having the sequence shown in SEQ ID NO: 15; and
a VL FW4 having the sequence shown in SEQ ID NO: 27.

11. The antibody or antibody fragment of claim 6 further comprising:
a VH FW1 having the sequence shown in SEQ ID NO: 23;
a VH FW2 having the sequence shown in SEQ ID NO: 10;
a VH FW3 having the sequence shown in SEQ ID NO: 31;
a VH FW4 having the sequence shown in SEQ ID NO: 25;
a VL FW1 having the sequence shown in SEQ ID NO: 13;
a VL FW2 having the sequence shown in SEQ ID NO: 14;
a VL FW3 having the sequence shown in SEQ ID NO: 15; and
a VL FW4 having the sequence shown in SEQ ID NO: 16.

12. The antibody or antibody fragment of claim 8, said antibody comprising:
(a) the VH FW1 having the amino acid sequence as shown in SEQ ID NO: 9;
the VH CDR1 having the sequence shown in SEQ ID NO: 1;
the VH FW2 having the sequence shown in SEQ ID NO: 10;
the VH CDR2 having the sequence shown in SEQ ID NO: 2;
the VH FW3 having the sequence shown in SEQ ID NO: 11;
the VH CDR3 having the sequence shown in SEQ ID NO: 3;
the VH FW4 having the sequence shown in SEQ ID NO: 12;
the VL FW1 having the sequence shown in SEQ ID NO: 13;
the VL CDR1 having the sequence shown in SEQ ID NO: 7;
the VL FW2 having the sequence shown in SEQ ID NO: 14;
the VL CDR2 having the sequence shown in SEQ ID NO: 5;
the VL FW3 having the sequence shown in SEQ ID NO: 15;
the VL CDR3 having the sequence shown in SEQ ID NO: 6;
and the VL FW4 having the sequence shown in SEQ ID NO: 16; or
(b) the VH FW1 having the sequence shown in SEQ ID NO: 9;
the VH CDR1 having the sequence shown in SEQ ID NO: 8;
the VH FW2 having the sequence shown in SEQ ID NO: 10;
the VH CDR2 having the sequence shown in SEQ ID NO: 2;
the VH FW3 having the sequence shown in SEQ ID NO: 19;
the VH CDR3 having the sequence shown in SEQ ID NO: 3;
the VH FW4 having the sequence shown in SEQ ID NO: 12;
the VL FW1 having the sequence shown in SEQ ID NO: 13;
the VL CDR1 having the sequence shown in SEQ ID NO: 7;
the VL FW2 having the sequence shown in SEQ ID NO: 14;
the VL CDR2 having the sequence shown in SEQ ID NO: 5;
the VL FW3 having the sequence shown in SEQ ID NO: 15;
the VL CDR3 having the sequence shown in SEQ ID NO: 6; and
the VL FW4 having the sequence shown in SEQ ID NO: 16;
(c) the VH FW1 having the sequence shown in SEQ ID NO: 17;
the VH CDR1 having the sequence shown in SEQ ID NO: 1;
the VH FW2 having the sequence shown in SEQ ID NO: 10;
the VH CDR2 having the sequence shown in SEQ ID NO: 2;
the VH FW3 having the sequence shown in SEQ ID NO: 18;
the VH CDR3 having the sequence shown in SEQ ID NO: 3;
the VH FW4 having the sequence shown in SEQ ID NO: 20;
the VL FW1 having the sequence shown in SEQ ID NO: 13;
the VL CDR1 having the sequence shown in SEQ ID NO: 7;
the VL FW2 having the sequence shown in SEQ ID NO: 14;
the VL CDR2 having the sequence shown in SEQ ID NO: 5;
the VL FW3 having the sequence shown in SEQ ID NO: 15;
the VL CDR3 having the sequence shown in SEQ ID NO: 6;
and the VL FW4 having the sequence shown in SEQ ID NO: 16; or
(d) the VH FW1 having the sequence shown in SEQ ID NO: 9;
the VH CDR1 having the sequence shown in SEQ ID NO: 8;
the VH FW2 having the sequence shown in SEQ ID NO: 10;
the VH CDR2 having the sequence shown in SEQ ID NO: 2;
the VH FW3 having the sequence shown in SEQ ID NO: 19;
the VH CDR3 having the sequence shown in SEQ ID NO: 3;
the VH FW4 having the sequence shown in SEQ ID NO: 12;
the VL FW1 having the sequence shown in SEQ ID NO: 13;
the VL CDR1 having the sequence shown in SEQ ID NO: 4;
the VL FW2 having the sequence shown in SEQ ID NO: 14;
the VL CDR2 having the sequence shown in SEQ ID NO: 5;
the VL FW3 having the sequence shown in SEQ ID NO: 15;
the VL CDR3 having the sequence shown in SEQ ID NO: 6; and
the VL FW4 having the sequence shown in SEQ ID NO: 16; or
(e) the VH FW1 having the sequence shown in SEQ ID NO: 9;
the VH CDR1 having the sequence shown in SEQ ID NO: 8;
the VH FW2 having the sequence shown in SEQ ID NO: 10;
the VH CDR2 having the sequence shown in SEQ ID NO: 2;
the VH FW3 having the sequence shown in SEQ ID NO: 19;
the VH CDR3 having the sequence shown in SEQ ID NO: 3;
the VH FW4 having the sequence shown in SEQ ID NO: 12;
the VL FW1 having the sequence shown in SEQ ID NO: 21;
the VL CDR1 having the sequence shown in SEQ ID NO: 4;
the VL FW2 having the sequence shown in SEQ ID NO: 14;
the VL CDR2 having the sequence shown in SEQ ID NO: 5;
the VL FW3 having the sequence shown in SEQ ID NO: 15;
the VL CDR3 having the sequence shown in SEQ ID NO: 6; and
the VL FW4 having the sequence shown in SEQ ID NO: 22.

13. The antibody of any of claims 1-12, wherein the antibody is an IgA, IgG, IgE, IgA, IgD antibody.

14. The antibody of any of claims 1-12, wherein the antibody is a humanized antibody.

15. The antibody fragment of any of claims 1-12 wherein the antibody fragment is an Fab, scFv, Fv, F(ab′)₂, Fab₂, or Fab′ fragment.

16. A polynucleotide comprising a nucleic acid sequence encoding the VH domain and the VL domain of the antibody or antibody fragment of any one of claims 1-12.

17. A vector comprising the polynucleotide of claim 16.

18. A cell comprising the vector of claim 17.

19. A method of expressing an antibody comprising: culturing the cell of claim 18 under conditions where said cell expresses the antibody; and harvesting the expressed antibody.

20. A method for detecting ovarian cancer cells in a patient comprising:
contacting a patient sample with the antibody or antibody fragment of any of claims 1-12;
detecting presence or absence of binding between the antibody or antibody fragment and ovarian cancer cells in the patient sample; and
detecting presence of the ovarian cancer cells in the patient sample if binding is detected or determining absence of ovarian cancer cells in the patient sample if binding is not detected.

21. The method of claim 20, wherein the antibody or antibody fragment further comprises a detectable label.

22. A method for detecting myeloma cells in a patient comprising:
contacting a patient sample with the antibody or antibody fragment of any of claims 1-12;
detecting presence or absence of binding between the antibody or antibody fragment and myeloma cells in the patient sample; and
detecting presence of the myeloma cells in the patient sample if binding is detected or determining absence of myeloma cells in the patient sample if binding is not detected.

23. The method of claim 22, wherein the antibody or antibody fragment further comprises a detectable label.

24. A method for diagnosing ovarian cancer in a patient comprising:
contacting a patient sample with the antibody or antibody fragment of any of claims 1-12;
detecting presence or absence of binding between the antibody or antibody fragment and ovarian cancer cells in a patient sample; and
diagnosing the patient with ovarian cancer if binding is detected.

25. The method of claim 24, wherein the antibody or antibody fragment further comprises a detectable label.

26. A method for diagnosing myeloma in a patient comprising:
contacting a patient sample with the antibody or antibody fragment of any of claims 1-12;
detecting presence or absence of binding between the antibody or antibody fragment and myeloma cells in a patient sample; and
diagnosing the patient with myeloma if binding is detected.

27. The method of claim 26, wherein the antibody or antibody fragment further comprises a detectable label.

28. A method for treating a patient with ovarian cancer comprising:
administering an effective amount of any of the antibodies or antibody fragments of any of claims 1-12 to an ovarian cancer patient.

29. The method of claim 28, wherein the antibody or antibody fragment further comprises a therapeutic agent.

30. The method of claim 29, wherein the antibody or antibody fragment is conjugated to the therapeutic agent.

31. A method for treating a patient with myeloma comprising:
administering an effective amount of any of the antibodies or antibody fragments of any of claims 1-12 to a myeloma patient.

32. The method of claim 31, wherein the antibody or antibody fragment further comprises a therapeutic agent.

33. The method of claim 32, wherein the antibody or antibody fragment is conjugated to the therapeutic agent.

34. A method for killing or inhibiting growth of ovarian cancer cells comprising:
contacting the ovarian cancer cells with any of the antibodies or antibody fragments of any of claims 1-12 in an amount sufficient to kill or inhibit growth of the ovarian cancer cells.

35. The method of claim 34, wherein the antibody or antibody fragment further comprises a therapeutic agent.

36. The method of claim 35, wherein the antibody or antibody fragment is conjugated to the therapeutic agent.

37. A method for killing or inhibiting growth of myeloma cells comprising:
contacting the myeloma cells with any of the antibodies or antibody fragments of any of claims 1-12 in an amount sufficient to kill or inhibit growth of the myeloma cells.

38. The method of claim 37, wherein the antibody or antibody fragment further comprises a therapeutic agent.

39. The method of claim 38, wherein the antibody or antibody fragment is conjugated to the therapeutic agent.

40. A method for killing or inhibiting growth of myeloma cells comprising:
contacting the myeloma cells with any of the antibodies or antibody fragments of any of claims 1-12, in combination with an immunotherapeutic agent such as CAR-Tor CAR-NK.

41. The method of claim 40, wherein the antibody or antibody fragment further comprises a therapeutic agent.

42. The method of claim 41, wherein the antibody or antibody fragment is conjugated to the therapeutic agent.
